(19) United States
(12) Patent Application Publication
(10) Pub. No.: US 2011/0038823 A1
(43) Pub. Date: Feb. 17, 2011

(54) ANTIPERSPIRANT EMULSION PRODUCTS WITH IMPROVED EFFICACY AND PROCESSES FOR MAKING THE SAME

(75) Inventors: Brittany Phipps, Erie, CO (US); Thomas Doering, Scottsdale, AZ (US)

Correspondence Address: THE DIAL CORPORATION, 19001 N. Scottsdale Road, SCOTTSDALE, AZ, 85255, US

(73) Assignee: The Dial Corporation, Scottsdale, AZ (US)

(21) Appl. No.: 12/852,863

(22) Filed: Aug. 9, 2010

Related U.S. Application Data
(63) Continuation-in-part of application No. 12/540,532, filed on Aug. 13, 2009.

(51) Int. Cl. A61K 8/26; A61Q 15/00

(52) U.S. Cl. 424/68

(57) ABSTRACT

Antiperspirant emulsion products with improved antiperspirant efficacy and processes for making them are provided. In one embodiment, an antiperspirant emulsion product comprises a water-in-oil emulsion having a water phase and an oil phase. The water phase comprises an activated aluminum sesquichlorohydrate active system and the oil phase comprises cetyl PEG/PPG-10/1 dimethicone and a hydrophobic carrier.

### CROSS-REFERENCES TO RELATED APPLICATIONS

This is a continuation-in-part of application Ser. No. 12/540,532, filed Aug. 13, 2009, which is herein incorporated by reference in its entirety.

### FIELD OF THE INVENTION

The present invention generally relates to antiperspirant products and processes for making them, and more particularly relates to antiperspirant emulsion products with improved efficacy and processes for making them.

### BACKGROUND OF THE INVENTION

Antiperspirants and deodorants are popular personal care products used to prevent or eliminate perspiration and body odor caused by perspiration. Antiperspirants typically prevent the secretion of perspiration by blocking or plugging perspiration-secreting glands, such as those located at the underarms. Deodorants counteract or mask the unwanted odors caused by bacterial flora in secreted perspiration.

Antiperspirant sticks are desired by a large majority of the population because of the presence of antiperspirant active compounds that block or prevent the secretion of perspiration and the accompanying odors thereof and because of their ease of application. The antiperspirant product is applied to the skin by swiping or rubbing the stick across the skin, typically of the underarm. However, antiperspirant users often are disappointed in the chalky, brittle, and/or crumbly application of the stick across the skin. Deodorants, on the other hand, typically provide a better “glide” of the deodorant product across the underarm skin. The term “glide” typically is used to denote the friction between the antiperspirant and/or deodorant product and the skin. The smoother the glide, or the less friction between the product and the skin, the more desirable the product is to users. While deodorants typically exhibit smoother glide than antiperspirant sticks, they do not prevent or eliminate the secretion of perspiration as do antiperspirants because they do not contain active antiperspirant compounds. Active antiperspirant compounds generally cannot be added to deodorants because the alkalinity of the deodorants cause the antiperspirant compounds, typically acidic, to precipitate or settle out of deodorants. Thus, there is a need for antiperspirant products that exhibit antiperspirant efficacy with the feel of deodorants.

Antiperspirant emulsion formulas that exhibit the feel of deodorants, with minimal crumbling or caking, are known. Such products typically include an emulsion of an aqueous phase containing an active antiperspirant compound, such as an antiperspirant metal salt, and an oil phase containing, for example, a volatile silicone, fragrances, gellants, and other additives. While such formulas may provide the preferred glide of deodorants, conventional antiperspirant emulsions do not exhibit the antiperspirant efficacy that solid wax antiperspirant sticks do. Once introduced into the water phase, the antiperspirant salt experiences at least some loss of functionality.

Accordingly, it is desirable to provide antiperspirant emulsion products that exhibit improved antiperspirant efficacy and that have the feel of deodorants. In addition, it is desirable to provide processes for making such antiperspirant emulsion products. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Antiperspirant emulsion products with improved antiperspirant efficacy and processes for making them are provided. In accordance with one exemplary embodiment, an antiperspirant emulsion product comprises a water-in-oil emulsion having a water phase and an oil phase. The water phase comprises an activated aluminum sesquichlorohydrate active system and the oil phase comprises cetyl PEG/PPG-10/1 dimethicone and a hydrophobic carrier.

In accordance with another exemplary embodiment, a process for making an antiperspirant emulsion product comprises combining water and an aluminum sesquichlorohydrate active system to form a water phase and activating the aluminum sesquichlorohydrate active system. A carrier, a structurant, and cetyl PEG/PPG-10/1 dimethicone are combined, the structurant is melted, and an oil phase is formed. The water phase and the oil phase are mixed.

In accordance with a further exemplary embodiment, an antiperspirant emulsion product comprises a water-in-oil emulsion having a water phase and an oil phase. The water phase comprises an activated aluminum sesquichlorohydrate active system, propylene glycol, and water. The oil phase comprises cetyl PEG/PPG-10/1 dimethicone, C₁₂-C₁₅ alkyl benzoate, high molecular weight polyethylene, synthetic wax, dimethicone, and, optionally, cyclohexasiloxane.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The various embodiments contemplated herein relate to antiperspirant emulsion products with improved antiperspirant efficacy. The various embodiments of the antiperspirant emulsion products comprise an aluminum sesquichlorohydrate active system, described in more detail below. During the manufacturing of the antiperspirant emulsion products contemplated herein, the water phase comprising the aluminum sesquichlorohydrate active system is heated to relatively high temperatures, that is, about 75 to about 80° C. It unexpectedly has been found that, when heated to such high temperatures, the aluminum sesquichlorohydrate active system is “activated,” as described in more detail below, which increases the antiperspirant efficacy of the active system and, thus, the antiperspirant efficacy of a subsequently-formed antiperspirant emulsion product.

In addition, the various embodiments of the antiperspirant emulsion products comprise cetyl PEG/PPG-10/1 dimethicone as an emulsifier. It has been unexpectedly found that the use of cetyl PEG/PPG-10/1 dimethicone as an emulsifier in an antiperspirant product causes the antiperspirant product to exhibit skin feel characteristics that are typical of deodorant sticks. For example, with cetyl PEG/PPG-10/1 dimethicone, the antiperspirant emulsion products glide onto skin with less friction, that is, in a smoother manner, than conventional antiperspirants while still maintaining a solid consistency for easy application. The various embodiments of the antiperspirant emulsion products also comprise water and volatile carrier fluids that provide a cooling effect to the skin when they evaporate upon application to the skin.

In this regard, in one exemplary embodiment of the present invention, the antiperspirant emulsion product is a water-in-oil emulsion comprising a water phase mixed with an oil phase. Preferably, the antiperspirant emulsion product comprises a water phase in an amount of about 60 to about 85 weight percent (wt. %) of the total antiperspirant emulsion product and an oil phase in an amount of about 15 to about 40 wt. % of the total antiperspirant emulsion product. The antiperspirant emulsion product preferably has a soft, non-flowing, solid composition that can be rubbed or wiped across the skin, particularly the underarm. However, the various embodiments are not so limited and the antiperspirant product can also have a gel, cream, or lotion consistency.

As mentioned above, the water phase of the antiperspirant emulsion product comprises a water-soluble “activated” aluminum sesquichlorohydrate active system. The aluminum sesquichlorohydrate active system contains aluminum chlorohydrate, calcium chloride, water, and glycine. In one exemplary embodiment, the aluminum sesquichlorohydrate active system comprises aluminum chlorohydrate in an amount of about 30 to 35 wt.% of the total active system. In one embodiment, the aluminum sesquichlorohydrate active system is present in an amount of about 60 to about 90 wt. % of the total the water phase of the antiperspirant emulsion product.

The active ingredients in the aluminum sesquichlorohydrate active system, that is, the aluminum species, are thought to reduce sweating by diffusing through the sweat ducts of the apocrine glands (sweat glands responsible for body odor) and hydrolyzing in the sweat ducts, where they combine with proteins to form an amorphous aluminum hydroxide agglomerate, plugging the sweat ducts so perspiration can not diffuse to the skin surface. While it would be expected that an antiperspirant active system comprising only an aluminum salt would be less efficacious than a conventional active system comprising aluminum and zirconium salts, it unexpectedly has been found that, when heated during manufacture to relatively high temperatures in the range of about 75 to about 80° C., the aluminum sesquichlorohydrate active system is “activated.” In this regard, at these high temperatures, larger aluminum species of the active system are transformed into smaller aluminum species having lower molecular weights. The smaller aluminum species can more easily diffuse through the sweat ducts, thus providing improved antiperspirant efficacy compared to conventional, “non-activated” antiperspirant salts. Accordingly, as used herein, the term “activated aluminum sesquichlorohydrate active system” means an aluminum sesquichlorohydrate active system that has be heated during manufacture of an antiperspirant emulsion product to temperatures sufficient to transform aluminum species having higher molecular weights to aluminum species having lower molecular weights.

As noted above, the aluminum sesquichlorohydrate active system also comprises calcium chloride. Calcium chloride serves as an additional activator for the aluminum chlorohydrate. In one exemplary embodiment, the aluminum sesquichlorohydrate active system comprises calcium chloride in an amount of about 5 to about 7 wt. % of the total active system. Glycine is also present in the aluminum sesquichlorohydrate active system. It was unexpectedly found that glycine helps to maintain the activation status of aluminum sesquichlorohydrate over extended periods of time. In one exemplary embodiment, the aluminum sesquichlorohydrate active system comprises glycine in an amount of about 2 to about 5 wt. % of the total active system. In another embodiment, the aluminum sesquichlorohydrate active system also comprises water as a carrier in an amount of about 53 to about 63 wt. % of the total active system. With this relatively large amount of water, the aluminum chlorohydrate is in a solution even before being added to the water phase during manufacturing, thus making it easier to form a homogeneous water phase than if a conventional antiperspirant salt were used to make the antiperspirant emulsion product.

In accordance with another exemplary embodiment, the water phase also comprises at least one water soluble carrier/solubilizer present in a sufficient amount to solubilize or disperse the water phase ingredients of the antiperspirant emulsion product. Such carriers/solubilizers suitable for use in the antiperspirant product include, but are not limited to, propylene glycol, glycerol, dipropyl glycol, ethylene glycol, butylene glycol, propylene carbonate, dimethyl isosorbide, hexylene glycol, ethanol, n-butyl alcohol, n-propyl alcohol, isopropyl alcohol, and the like. In a preferred embodiment, the water phase comprises propylene glycol and, in a more preferred embodiment, the water phase comprises propylene glycol in an amount of about 8 to about 15 wt. % of the total water phase. In addition to the carrier/solubilizer, the antiperspirant product may comprise water additional to the water in the aluminum sesquichlorohydrate active system. As noted above, the water evaporates from the antiperspirant product upon application of the antiperspirant emulsion product to the skin, providing a cooling sensation to the skin.

As noted above, the oil phase of the antiperspirant product comprises an emulsifier of cetyl PEG/PPG-10/1 dimethicone, in accordance with an exemplary embodiment. Cetyl PEG/PPG-10/1 dimethicone is a copolymer of cetyl dimethicone and an alkoxylated derivative of dimethicone containing an average of 10 moles of ethylene oxide and 1 mole of propylene oxide. The use of cetyl PEG/PPG-10/1 dimethicone as an emulsifier in the antiperspirant emulsion product causes the antiperspirant emulsion product to exhibit skin feel characteristics that are typical of deodorant sticks. For example, with cetyl PEG/PPG-10/1 dimethicone, the antiperspirant emulsion products glide onto the skin with less friction while still maintaining a solid consistency for easy application. In addition, when applied, the antiperspirant products are moister than typical antiperspirants and thus give the skin a moister and softer feel. In a preferred embodiment, the oil phase comprises cetyl PEG/PPG-10/1 dimethicone in an amount of about 3 to about 7 wt. % of the total oil phase.

Further included in the oil phase of the antiperspirant product is at least one structurant and/or gellant (hereinafter referred to collectively as “structurant”) that facilitates the solid consistency of the antiperspirant emulsion product. Naturally-occurring or synthetic waxy materials or combinations thereof can be used as such structurants. Examples of these waxy materials include those fatty alcohols that are solid at room temperature and hydrocarbon waxes or silicone waxes. Such materials are widely available, and by suitable selection of the materials themselves and their concentrations in the formulation, it is possible to obtain either a soft solid or a firm solid. In a preferred embodiment, the oil phase comprises a high molecular weight (MW) polyethylene. As used herein, the term “high molecular weight polyethylene” or “high MW polyethylene” means polyethylene having a molecular weight of 200 to 5000 daltons (Da). In a more preferred embodiment, the oil phase comprises high MW polyethylene having a molecular weight of about 500 Da. In another preferred embodiment, the oil phase comprises high MW polyethylene in an amount of about 30 to about 60 wt. % of the total oil phase. In this regard, polyethylene can be used in smaller amounts as a structurant in the antiperspirant emulsion products than other structurants, such as stearyl alcohol, that can exhibit undesirable properties in the antiperspirant product. Stearyl alcohol is commonly used as a structurant in solid stick underarm products. However, stearyl alcohol has a tendency to leave visible white deposits on the skin, and the deposits can also transfer onto clothing when the clothing comes into contact with the skin. Accordingly, in another preferred embodiment, the oil phase comprises substantially no stearyl alcohol. The term “substantially no stearyl alcohol” as used herein means no stearyl alcohol or stearyl alcohol in an amount that is sufficiently small so that it would not cause visible white residue to deposit on skin and/or clothing after application of the antiperspirant product to the skin.

In accordance with another exemplary embodiment, when high MW polyethylene is used in the oil phase as a structurant, the oil phase also comprises at least one low MW synthetic wax. In addition to facilitating the high MW polyethylene by serving as a structurant, the low MW synthetic wax also improves the manufacturing processes of the antiperspirant products. Generally, polyethylene has a relatively high melting point (70-100° C.) and, thus, as described in more detail below, the oil phase of the antiperspirant product must be heated to this high melting point to melt the polyethylene. However, this high temperature heating may result in higher manufacturing costs and unpredictable and/or non-repeatable yields of the antiperspirant product. The presence of an effective amount of low MW synthetic wax (synthetic wax having a molecular weight in the range of 1200-2900 Da) modifies the high MW polyethylene, lowering the melting point of the polyethylene. Accordingly, the presence of the low MW synthetic wax may result in lower manufacturing costs and higher yield of the antiperspirant product. In an exemplary embodiment, the low MW synthetic wax is present in the oil phase in an amount of about 0 to about 1 wt. % of the total oil phase. In a preferred embodiment, the low MW synthetic wax has a molecular weight of about 1800 Da.

In addition to improving hardness of the antiperspirant stick product, the low MW synthetic wax reduces syneresis and tackiness and also has a high refractive index (R.I.) that minimizes and/or prevents a white residue on the skin by masking the antiperspirant metallic salt(s) that stays upon the skin upon evaporation of the carrier(s). As used herein, the term “high refractive index” means a refractive index no less than about 1.4. While the use of low MW synthetic wax to lower the melting point of high MW polyethylene is described herein in the context of an antiperspirant product comprising cetyl PEG/PPG-10/1 dimethicone as an emulsifier, it will be appreciated that low MW synthetic wax can be used to lower the melting point of high MW polyethylene in an antiperspirant emulsion product containing any suitable emulsifier.

The oil phase further comprises at least one hydrophobic carrier. An example of suitable hydrophobic carriers includes liquid siloxanes and particularly volatile polyorganosiloxanes, that is, liquid materials having a measurable vapor pressure at ambient conditions. The polyorganosiloxanes can be linear or cyclic or mixtures thereof. Preferred siloxanes include cyclomethicones, such as cyclotetrasiloxane, cyclopentasiloxane and cyclohexasiloxane, and mixtures thereof. The carrier also may comprise, additionally or alternatively, nonvolatile silicones such as dimethicone and dimethicone copolyols. Examples of suitable dimethicone and dimethicone copolyols include polyalkyl siloxanes, polyalkylaryl siloxanes, and polyether siloxane copolymers.

The oil phase may also comprise a high R.I. hydrophobic compound. The high R.I. hydrophobic compound minimizes and/or prevents a white residue on the skin by masking the antiperspirant metallic salt that stays upon the skin upon evaporation of the carrier(s). Examples of high R.I. hydrophobic compounds for use in the antiperspirant products include C₁₂-C₁₅ alkyl benzoate, such as Finsolv TN® available from Innospec of the United Kingdom, PPG-14 butyl ether, and phenyl trimethicone. In a preferred embodiment, the oil phase comprises C₁₂-C₁₅ alkyl benzoate and, in a more preferred embodiment, the oil phase comprises C₁₂-C₁₅ alkyl benzoate in an amount of about 15 to about 40 wt. % of the total oil phase of the antiperspirant emulsion product.

In addition to the ingredients identified above, the antiperspirant product may comprise additives, such as those used in conventional antiperspirants. These additives include, but are not limited to, fragrances, including encapsulated fragrances, dyes, pigments, preservatives, antioxidants, moisturizers, and the like. These optional ingredients can be included in the antiperspirant emulsion product in an amount of 0 to about 20 wt. %.

The antiperspirant emulsion product according to various embodiments can be prepared by combining the ingredients of the water phase in a mixing vessel using sufficient agitation to prepare a homogeneous mixture. During formation of the homogeneous water phase or after the homogeneous water phase is formed, the water phase is heated to a temperature in the range of about 75 to about 80° C. The water phase is heated for a time sufficient to activate the aluminum sesquichlorohydrate, for example, for about ten minutes to about one hour or more. As noted above, while the water phase is heated at these temperatures, the aluminum sesquichlorohydrate active system is activated. Before, during, or after formation of the water phase, the ingredients of the oil phase are combined and mixed thoroughly. During formation of the oil phase, or after the oil phase is formed, the oil phase is heated to a temperature at least sufficient to melt the structurant. In one embodiment, the oil phase is heated to a temperature in the range of about 90 to about 95° C. until the structurant (i.e., high MW polyethylene) is melted. Heat then is removed from the oil phase, which is allowed to cool to about 75 to about 80° C., while under significant agitation. Next, the oil phase and the water phase are mixed together using any known method to form a water-in-oil emulsion. In one exemplary embodiment, the water phase is transferred to the oil phase via droplet to form an emulsion, keeping both separated phases mixing and at the same temperatures, preferably about 75 to about 80° C. Once the water phase is fully transferred to the oil phase, rapid mixing of the emulsion is maintained for a time sufficient to stabilize the emulsion. In one embodiment, the rapid mixing is continued for about 10 minutes. The emulsion is cooled to about 70 to about 75° C. Any additive, such as fragrance, is then added to the water-in-oil emulsion. The resulting liquid antiperspirant emulsion product is poured into suitable molds and then cooled to room temperature so that the emulsion solidifies.

The following is an exemplary embodiment of an antiperspirant emulsion product, with each of the components set forth in weight percent of their respective phases. The example is provided for illustration purposes only and is not meant to limit the various embodiments of the antiperspirant product in any way.

### EXAMPLE

| Water Phase | | Oil Phase | |
|---|---|---|---|
| Component | wt. % | Component | wt. % |
| Aluminum | | C₁₂-C₁₅ Alkyl Benzoate | 15-40 |
| Sesquichlorohydrate | | Synthetic wax (MW = 1800) | 0-1 |
| Active System | 60-90 | Polyethylene (MW = 500) | 30-60 |
| Propylene glycol | 8-15 | Cetyl PEG/PPG-10/ | 3-7 |
| | | 1 dimethicone | |
| Water | q.s. | Dimethicone | 0-7 |
| | | Cyclohexasiloxane | 0-30 |
| Total Water Phase | 60-85 | Total Oil Phase | 15-40 |

Fragrance 0-3
Total 100

Accordingly, antiperspirant emulsion products with improved antiperspirant efficacy have been provided. In this regard, the various embodiments of the antiperspirant emulsion products comprise an activated aluminum sesquichlorohydrate active system and cetyl PEG/PPG-10/1 dimethicone as an emulsifier. During the manufacturing of the antiperspirant emulsion products contemplated herein, the aluminum sesquichlorohydrate active system is heated to relatively high temperatures. It unexpectedly has been found that, when heated to high temperatures, the aluminum sesquichlorohydrate active system is “activated,” which increases the antiperspirant efficacy of the active system and, thus, the antiperspirant efficacy of a subsequently-formed antiperspirant emulsion product. Use of cetyl PEG/PPG-10/1 dimethicone as an emulsifier in the antiperspirant emulsion products causes the antiperspirant emulsion products to exhibit skin feel characteristics that are typical of deodorant products.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant emulsion product comprising a water-in-oil emulsion having a water phase and an oil phase, the water phase comprising an activated aluminum sesquichlorohydrate active system and the oil phase comprising cetyl PEG/PPG-10/1 dimethicone and a hydrophobic carrier.

2. The antiperspirant emulsion product of claim 1, wherein the water phase is present in an amount of about 60 to about 85 wt. % of the antiperspirant emulsion product.

3. The antiperspirant emulsion product of claim 1, wherein the oil phase is present in an amount of about 15 to about 40 wt. % of the antiperspirant emulsion product.

4. The antiperspirant emulsion product of claim 1, wherein the activated aluminum sesquichlorohydrate active system is present in an amount of about 60 to about 90 wt. % of the water phase.

5. The antiperspirant emulsion product of claim 1, wherein the activated aluminum sesquichlorohydrate active system comprises aluminum chlorohydrate, calcium chloride, additional water, and glycine.

6. The antiperspirant emulsion product of claim 5, wherein the aluminum chlorohydrate is present in an amount of about 30 to about 35 wt. % of the activated aluminum sesquichlorohydrate active system.

7. The antiperspirant emulsion product of claim 5, wherein the calcium chloride is present in an amount of about 5 to about 7 wt. % of the activated aluminum sesquichlorohydrate active system.

8. The antiperspirant emulsion product of claim 5, wherein the additional water is present in an amount of about 53 to about 63 wt. % of the activated aluminum sesquichlorohydrate active system.

9. The antiperspirant emulsion product of claim 5, wherein the glycine is present in an amount of about 2 to about 5 wt. % of the activated aluminum sesquichlorohydrate active system.

10. The antiperspirant emulsion product of claim 1, wherein the cetyl PEG/PPG-10/1 dimethicone is present in an amount of about 3 to about 7 wt. % of the oil phase.

11. The antiperspirant emulsion product of claim 1, wherein the hydrophobic carrier is a cyclomethicone.

12. The antiperspirant emulsion product of claim 1, wherein the oil phase further comprises a high molecular weight polyethylene.

13. A process for making an antiperspirant emulsion product, the process comprising the steps of:
combining water and an aluminum sesquichlorohydrate active system to form a water phase;
activating the aluminum sesquichlorohydrate active system;
combining a carrier, a structurant, and cetyl PEG/PPG-10/1 dimethicone;
melting the structurant and forming an oil phase;
mixing the water phase and the oil phase; and
solidifying the emulsion.

14. The process of claim 13, wherein the step of activating comprises heating the aluminum sesquichlorohydrate active system to temperatures that result in a transformation of aluminum species having higher molecular weights to aluminum species having lower molecular weights.

15. The process of claim 14, wherein the step of heating comprises heating the aluminum sesquichlorohydrate active system to temperatures in a range of about 75 to about 80° C.

16. The process of claim 13, wherein the step of mixing comprises combining the water phase and the oil phase so that the water phase is present in the antiperspirant emulsion product in an amount of about 60 to about 85 wt. % of the antiperspirant emulsion product and the oil phase is present in the antiperspirant emulsion product in an amount of about 15 to about 40 wt. % of the antiperspirant emulsion product.

17. The process of claim 13, wherein the step of combining water and an aluminum sesquichlorohydrate active system comprises adding the aluminum sesquichlorohydrate active system so that it is present in the antiperspirant emulsion product in an amount of about 60 to about 90 wt. % of the water phase.

18. The process of claim 13, wherein the step of combining water and an aluminum sesquichlorohydrate active system comprises adding the aluminum sesquichlorohydrate active system comprising aluminum chlorohydrate, calcium chloride, additional water, and glycine.

19. The process of claim 13, wherein the step of combining a carrier, structurant, and cetyl PEG/PPG-10/1 dimethicone comprises adding cetyl PEG/PPG-10/1 dimethicone so that it is present in the antiperspirant emulsion product in an amount of about 3 to about 7 wt. % of the oil phase

20. The process of claim 13, wherein the step of combining a carrier, structurant, and cetyl PEG/PPG-10/1 dimethicone comprises adding a cyclomethicone as the carrier.

21. The process of claim 13, wherein the step of combining a carrier, structurant, and cetyl PEG/PPG-10/1 dimethicone comprises adding a high molecular weight polyethylene as the structurant.

22. An antiperspirant emulsion product comprising a water-in-oil emulsion having a water phase and an oil phase, the water phase comprising an activated aluminum sesquichlorohydrate active system, propylene glycol, and water and the oil phase comprising cetyl PEG/PPG-10/1 dimethicone, C₁₂-C₁₅ alkyl benzoate, high molecular weight polyethylene, synthetic wax, dimethicone, and, optionally, cyclohexasiloxane.

23. The antiperspirant emulsion product of claim 22, wherein the water phase is present in an amount of about 60 to about 85 wt. % of the antiperspirant emulsion product and the oil phase is present in an amount of about 15 to about 40 wt. % of the antiperspirant emulsion product.

24. The antiperspirant emulsion product of claim 22, wherein the activated aluminum sesquichlorohydrate active system is present in an amount of about 60 to about 90 wt. % of the water phase.
